(19) United States
(12) Patent Application Publication
(10) Pub. No.: US 2011/0218647 A1
(43) Pub. Date: Sep. 8, 2011

(54) MEDICAL IMPLANT

(76) Inventor: Donald Eagland, Huddersfield Yorkshire (GB)

(21) Appl. No.: 12/737,974

(22) PCT Filed: Jul. 14, 2009
(86) PCT No.: PCT/GB2009/050854

§ 371 Date: May 19, 2011

(30) Foreign Application Priority Data
Jul. 16, 2008 (GB) 0812940.5

(51) Int. Cl. A61F 2/02

(52) U.S. Cl. 623/23.72

(57) ABSTRACT

A cartilage plug (14) comprises cross-linked polyvinylalcohol, a polyvinylalcohol fibre and fumed silica. The plug may also include chondrocytes and/or hyaluronic acid.

This invention relates to a medical implant and particularly, although not exclusively, relates to the replacement of defective natural cartilage, where the defects are caused by traumatic injury which causes acute damage to the cartilage and/or by disease or the long term effects of unrepaired cartilage injuries which, over a prolonged period of time, cause a chronic deterioration of the cartilage.

Human cartilage is one of the few vascular tissues in the body. It serves to prevent bone growth into the articulating surface of joints which would otherwise interfere with the motion of joints. Cartilage is semi-permeable and receives its nutrients from the synovial fluid which surrounds cartilaginous tissue in articulating joints and which diffuses into the cartilage during motion of the joint. Cartilage itself also possesses visco-elastic and lubricating properties. Accordingly, any material which is proposed for use in the repair or replacement of natural cartilage must possess physical and mechanical properties which are as close as possible or exceed those of natural cartilage.

The playing of sports can result in accidents which cause traumatic injury to cartilage, particularly that surrounding the knees, elbows and shoulders. In addition, persons may be inflicted with arthritic diseases which may cause degeneration of cartilage. Osteoarthritis may set in following a traumatic injury to cartilage which is not repaired or is repaired improperly.

When a cartilage defect is caused by traumatic injury and is extensive enough in size to involve a large mass of cartilage, the damage is not capable of self healing.

To address the problem of cartilage damage, in some cases, if the extent of the damage is only relatively minor, a decision may be taken to take no action; however, where damage is more extensive complete joint replacement may be undertaken.

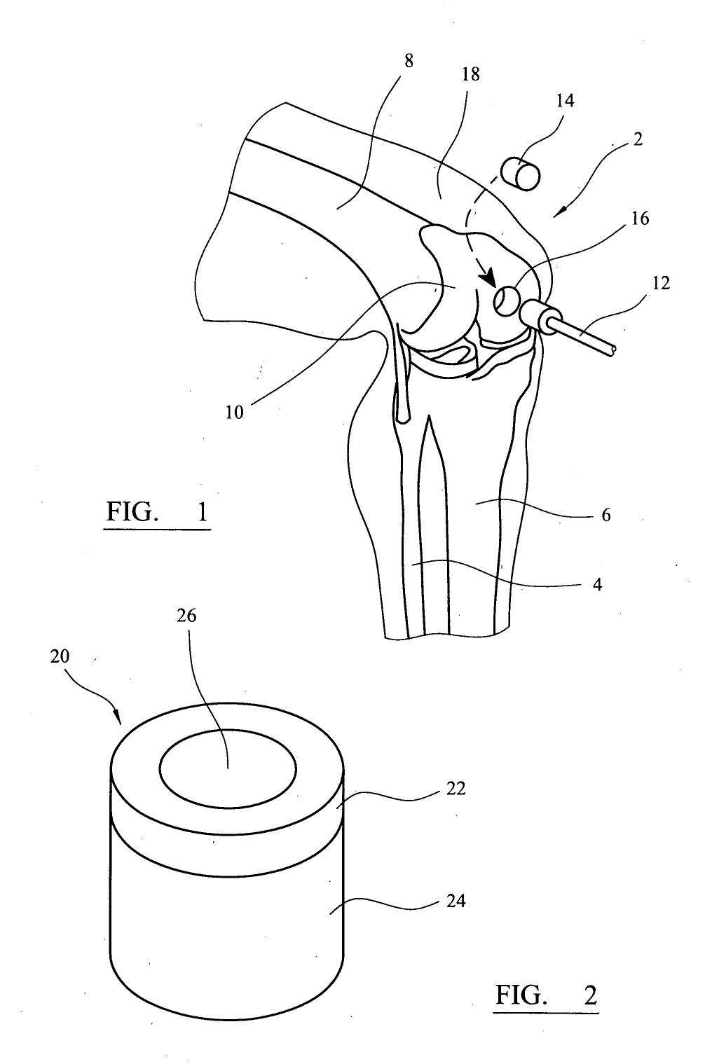

It is known to treat chondral or oseochondral defects of articular cartilage surfaces by use of a cartilage repair plug as shown in FIG. 1 of the accompanying drawings which shows a schematic representation of a knee joint. The joint 2 includes an associated fibia 4, tibia 6 and femur 8, coated with articular cartilage 10. To repair a defect in the cartilage 10, the damage is cored out using a suitable corer 12 to define a cylindrical opening 16 into which a cartilage plug 14 may be fitted as represented by arrow 18. The plug protrudes slightly from the surface so that some load incident on the joint is absorbed by the plug 14.

Known cartilage plugs may disadvantageously work lose within their openings or simply have relatively poor mechanical properties, both initially and over time. As a result the plugs may wear and/or fail prematurely.

It is an object of the present invention to address problems associated with medical implants, for example as described above.

According to a first aspect of the invention, there is provided a medical implant comprising a polymeric material and a fibrous filler.

Said medical implant suitably includes at least 40 wt %, preferably at least 50 wt %, more preferably at least 60 wt %, especially at least 65 wt % water. In some cases, said medical implant may include 70 wt % or more water. The amount of water in the implant is preferably less than 85 wt % and may be 80 wt % or less.

Said polymeric material preferably comprises, more preferably consists essentially of a hydrogel.

Said hydrogel preferably comprises an optionally derivatised, for example cross-linked, hydrophilic polymer. The hydrophilic polymer may include relatively hydrophilic regions and relatively hydrophobic regions.

Said hydrophilic polymer may comprise optionally-derivatised e.g. cross-linked water soluble gums, for example gum arabic, karaya gum, tragacanth gum, ghatti gum, guar gum; soybean derivatives, for example locust bean gum, tamarind gum; water soluble biopolymers, for example dextran, xanthan gum; water soluble proteins, for example gelatin type materials, carrageenan, agar and alginates, animal derivatives, casein, pectin; starch and starch derivatives, for example starch, modified starch, starch derivatives; cellulose derivatives, for example methyl cellulose, carboxymethyl cellulose, hydroxyethyl cellulose, hydroxypropyl cellulose; polyvinyls and maleic anhydride copolymers, for example polyvinyl alcohol, polyvinyl pyrrolidone; miscellaneous water soluble polyvinyls, for example maleic anhydride copolymers; polyacrylates and related systems, for example polyacrylates, polyacrylamides; polyimines and related systems, for example polyethylene oxides, polyethylenimines, polyethylene glycols; surface active water soluble polymers, for example lignosulfonates and related materials, lignites, tannins.

Preferred examples of suitable hydrophilic polymers include polymethacrylic acid polymers; polyimides; polyvinylalcohol and copolymers of the aforesaid.

Said hydrophilic polymer preferably includes a carbon atom containing backbone. The carbon atoms are preferably linked together by C—C single bonds. The backbone preferably includes no other types of atoms.

Said hydrophilic polymer preferably includes carbonyl moieties. Such moieties may be included in groups pendent from a backbone of the polymer. Said carbonyl moieties may be components of carboxylic acids or carboxylic acid derivates. Preferably carbonyl moieties are components of ester functional groups, for example groups —OCO—R¹⁰ wherein R¹⁰ represents an optionally-substituted alkyl or alkenyl moiety, especially a C₁₋₄ alkyl or alkenyl moiety. R¹⁰ is preferably an unsubstituted alkyl moiety especially a methyl group. Thus, said hydrophilic polymer preferably includes acetate moieties.

Said hydrophilic polymer preferably includes hydroxyl groups which are suitably pendent from a backbone of the polymer. Preferably hydroxyl groups are bonded directly to the backbone, preferably carbon atoms thereof. Preferred hydroxy groups comprise alcohol functional groups.

Said hydrophilic polymer preferably includes both carbonyl moieties as described and hydroxyl moieties as described, wherein suitably the carbonyl moieties and hydroxyl moieties are present in separate functional groups pendent from the polymer backbone.

Suitably at least 50 mole %, preferably at least 75 mole %, more preferably at least 95 mole %, especially about 100 mole % of said hydrophilic polymer is made up of repeat units which include functional groups which include carbonyl moieties (preferably as part of carboxylic acid or carboxylic acid derivative functional groups) or hydroxyl (especially alcohol) moieties. Suitably, the sum of the mole % of carbonyl containing functional group (e.g. carboxylic acid or carboxylic acid derivative functional groups) and hydroxyl (especially alcohol) functional groups in said hydrophilic polymer is at least 70 mole %, preferably at least 90 mole %, more preferably at least 95 mole %, especially about 100 mole %. Thus, in a preferred embodiment, an hydrophilic polymer material which includes the aforementioned functional groups is not a copolymer which includes other types of functional groups.

Said hydrophilic polymer preferably comprises a polyvinyl polymer. Suitably the sum of the mole % of vinyl moieties in said polymer is at least 70 mole %, preferably at least 90 mole %, more preferably at least 95 mole %, especially about 100 mole %.

The most preferred hydrogel comprises an optionally-derivatised, for example cross-linked, polyvinylalcohol. Preferred polyvinylalcohols include hydroxyl functional groups which are relatively hydrophilic and acetate functional groups which are relatively hydrophobic.

Said hydrogel preferably comprises an optionally-derivatised polyvinylalcohol which suitably consists essentially of vinylalcohol and vinyl acetate functional groups. Suitably, the polyvinylalcohol is hydrolyzed to an extent of less than 100 mole %, preferably less than 95 mole %. It may be hydrolysed to an extent of at least 10 mole %, preferably at least 25 mole %, more preferably at least 50 mole %, especially at least 60 mole %. Suitably, in said polyvinylalcohol, the ratio of the mole % of vinylalcohol moieties to vinylacetate moieties is at least 0.5, preferably at least 1, more preferably at least 3. The ratio may be less than 10, preferably less than 8.

Preferred polyvinylalcohols have a viscosity (measured on a 4% aqueous solution at 20° C.) of at least 2 mPa·s, preferably at least 4 mPa·s. The viscosity may be less than 100 mPa·s, preferably less than 75 mPa·s.

Said hydrophilic polymer of said hydrogel is preferably cross-linked by a cross-linking means.

A preferred cross-linking means comprises a chemical cross-linking material. Such a material is preferably a polyfunctional compound having at least two functional groups capable of reacting with functional groups of said hydrophilic polymer. Preferably, said cross-linking material includes one or more of carbonyl, carboxyl, hydroxy, epoxy, halogen or amino functional groups which are capable of reacting with groups present along the polymer backbone or in the polymer structure of the hydrophilic polymer. Preferred cross-linking materials include at least two aldehyde groups. Thus, in a preferred embodiment, said hydrogel includes a material formed by cross-linking polyvinylalcohol using a material having at least two aldehyde groups. Thus, said hydrogel may include a moiety of formula I.

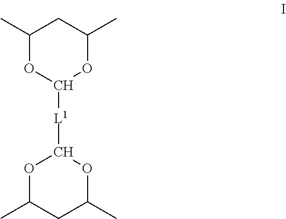

wherein L¹ is a residue of said cross-linking material.

Said cross-linking material preferably comprises a second polymeric material. Said second polymeric material preferably includes a repeat unit of formula

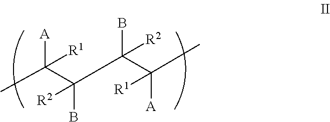

wherein A and B are the same or different, are selected from optionally-substituted aromatic and heteroaromatic groups and at least one comprises a relatively polar atom or group and R¹ and R² independently comprise relatively non-polar atoms or groups.

A and/or B could be multi-cyclic aromatic or heteroaromatic groups. Preferably, A and B are independently selected from optionally-substituted five or more preferably six-membered aromatic and heteroaromatic groups. Preferred heteroatoms of said heteroaromatic groups include nitrogen, oxygen and sulphur atoms of which oxygen and especially nitrogen, are preferred. Preferred heteroaromatic groups include only one heteroatom. Preferably, a or said heteroatom is positioned furthest away from the position of attachment of the heteroaromatic group to the polymer backbone. For example, where the heteroaromatic group comprises a six-membered ring, the heteroatom is preferably provided at the 4-position relative to the position of the bond of the ring with the polymeric backbone.

Preferably, A and B represent different groups. Preferably, one of A or B represents an optionally-substituted aromatic group and the other one represents an optionally-substituted heteroaromatic group. Preferably A represents an optionally-substituted aromatic group and B represents an optionally-substituted heteroaromatic group especially one including a nitrogen heteroatom such as a pyridinyl group.

Unless otherwise stated, optionally-substituted groups described herein, for example groups A and B, may be substituted by halogen atoms, and optionally substituted alkyl, acyl, acetal, hemiacetal, acetalalkyloxy, hemiacetalalkyloxy, nitro, cyano, alkoxy, hydroxy, amino, alkylamino, sulphinyl, alkylsulphinyl, sulphonyl, alkylsulphonyl, sulphonate, amido, alkylamido, alkylcarbonyl, alkoxycarbonyl, halocarbonyl and haloalkyl groups. Preferably, up to 3, more preferably up to 1 optional substituents may be provided on an optionally substituted group.

Unless otherwise stated, an alkyl group may have up to 10, preferably up to 6, more preferably up to 4 carbon atoms, with methyl and ethyl groups being especially preferred.

Preferably, A and B each represent polar atoms or group—that is, there is preferably some charge separation in groups A and B and/or groups A and B do not include carbon and hydrogen atoms only.

Preferably, at least one of A or B includes a functional group which can undergo a condensation reaction, for example on reaction with said hydrophilic polymer. Preferably, A includes a said functional group which can undergo a condensation reaction.

Preferably, one of groups A and B includes an optional substituent which includes a carbonyl or acetal group with a formyl group being especially preferred. The other one of groups A and B may include an optional substituent which is an alkyl group, with an optionally substituted, preferably unsubstituted, C₁₋₄ alkyl group, for example a methyl group, being especially preferred.

Preferably, A represents a group, for example an aromatic group, especially a phenyl group, substituted (preferably at the 4-position relative to polymeric backbone when A represents an optionally-substituted phenyl group) by a formyl group or a group of general formula

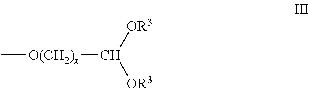

where x is an integer from 1 to 6 and each R³ is independently an alkyl or phenyl group or together form an alkalene group.

Preferably, B represents an optionally-substituted heteroaromatic group, especially a nitrogen-containing heteroaromatic group, substituted on the heteroatom with a hydrogen atom or an alkyl or aralkyl group. More preferably, B represents a group of general formula

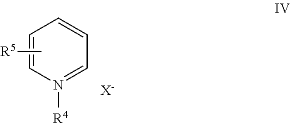

wherein R⁴ represents a hydrogen atom or an alkyl or aralkyl group, R⁵ represents a hydrogen atom or an alkyl group and X⁻ represents a strongly acidic ion. It may be an organic, for example alkyl, sulphate such a methylsulphate.

Preferably, R¹ and R² are independently selected from a hydrogen atom or an optionally-substituted, preferably unsubstituted, alkyl group. Preferably, R¹ and R² represent the same atom or group. Preferably, R¹ and R² represent a hydrogen atom.

Preferred second polymeric materials may be prepared from any of the following monomers by the method described in WO98/12239 and the content of the aforementioned document is incorporated herein by reference:

α-(p-formylstyryl)-pyridinium, γ-(p-formylstyryl)-pyridinium, α-(m-formylstyryl)-pyridinium, N-methyl-α-(p-formylstyryl)-pyridinium, N-methyl-β-(p-formylstyryl)-pyridinium, N-methyl-α-(m-formylstyryl)-pyridinium, N-methyl-α-(o-formylstyryl)-pyridinium, N-ethyl-α-(p-formylstyryl)-pyridinium, N-(2-hydroxyethyl)-α-(p-formylstyryl)-pyridinium, N-(2-hydroxyethyl)-γ-(p-formylstyryl)-pyridinium, N-allyl-α-(p-formylstyryl)-pyridinium, N-methyl-γ-(p-formylstyryl)-pyridinium, N-methyl-γ-(m-formylstyryl)-pyridinium, N-benzyl-α-(p-formylstyryl)-pyridinium, N-benzyl-γ-(p-formylstyryl)-pyridinium and N-carbamoylmethyl-γ-(p-formylstyryl)-pyridinium. These quaternary salts may be used in the form of hydrochlorides, hydrobromides, hydroiodides, perchlorates, tetrafluoroborates, methosulfates, phosphates, sulfates, methane-sulfonates and p-toluene-sulfonates.

Also, the monomer compounds may be styrylpyridinium salts possessing an acetal group, including the following:

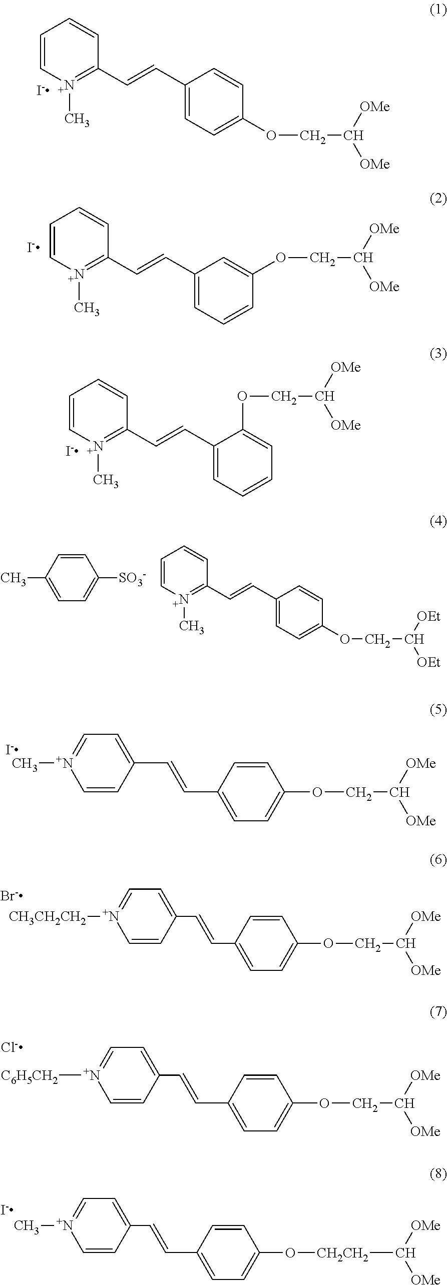

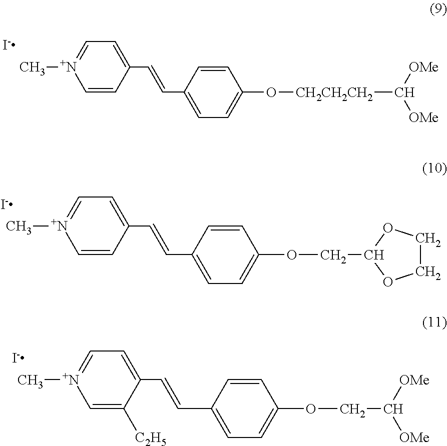

Thus, said second polymeric material is preferably prepared or preparable by providing a compound of general formula

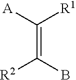

wherein A, B, R¹ and R² are as described above, in an aqueous solvent, (suitably so that molecules of said monomer aggregate) and causing the groups C═C in said compound to react with one another, (for example using UV radiation,) to form said second polymeric material.

Said second polymeric material may be of formula

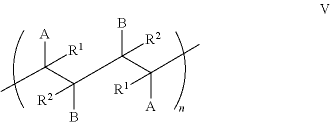

wherein A, B, R¹ and R² are as described above and n is an integer. Integer n is suitably 50 or less, preferably 20 or less, more preferably 10 or less, especially 5 or less. Integer n is suitably at least 1, preferably at least 2, more preferably at least 3.

Said hydrogel is preferably bio-compatible.

A said fibrous filler may comprise continuous or dis-continuous fibres. Said fibrous filler is preferably bio-compatible. It is preferably organic.

Said fibrous filler may comprise a spun fibre. It may be polymeric or non-polymeric. It preferably comprises an organic polymer.

Said fibrous filler may comprise an optionally cross-linked hydrophilic polymer. The hydrophilic polymer may independently have any of the properties and/or be as described herein for the optionally derivatised hydrophilic polymer of said hydrogel.

Said fibrous filler and said hydrogel may include respective functional groups which are the same. For example, both the fibrous filler and hydrogel may include hydroxyl groups.

Said hydrogel preferably includes an optionally cross-linked hydrophilic polymer and said fibrous filler preferably comprises the same optionally cross-linked hydrophilic polymer, although specific features, such as level of hydrolysis, molecular weight etc of the respective hydrophilic polymers may differ. Said hydrogel preferably comprises an optionally cross-linked polyvinylalcohol and said fibrous filler preferably independently comprises an optionally cross-linked (preferably non-cross-linked) polyvinylalcohol.

Said fibrous filler preferably comprises optionally derivatised polyvinylalcohol fibres. The polyvinylalcohol may be hydrolysed to an extent of at least 50 mole %, preferably at least 75 mole %, more preferably at least 90 mole %, especially at least 100 mole %. The polyvinylalcohol may be substantially fully hydrolysed.

Said fibrous filler preferably comprises fibres having average diameters of greater than 10 μm, greater than 20 μm, greater than 30 μm or greater than 40 μm. The average diameters may be less than 500 μm, less than 250 μm, less than 100 μm or less than 60 μm.

In a preferred embodiment, the average length of said fibres is at least 0.5 cm, preferably at least 1 cm. The average length may be less than 5 cm. Longer fibres may be used in some situations.

Said fibres are preferably bio-compatible.

The ratio of the weight of hydrogel on a dry matter basis (i.e. excluding water contained in it) to the weight of fibrous filler (on a dry matter basis) may be at least 1, is suitably at least 1.2, is preferably at least 1.4. Said ratio may be less than 3, suitably less than 2.5, preferably less than 2.0, more preferably less than 1.8.

The wt % of hydrogel in said medical implant (on a dry matter basis) is suitably at least 40 wt %, preferably at least 45 wt %. Said wt % may be less than 80 wt %, suitably less than 70 wt %, preferably less than 60 wt %, more preferably less than 55 wt %.

The wt % of fibrous filler in said medical implant (on a dry matter basis) is suitably at least 10 wt %, preferably at least 20 wt %, more preferably at least 25 wt %. In some cases, it may be at least 30 wt %. Said wt % may be less than 40 wt %, preferably less than 35 wt %.

The hydrogel may include at least 40 wt %, preferably at least 50 wt %, more preferably at least 60 wt %, especially at least 65 wt % water. The hydrogel may include less than 85 wt %, preferably less than 80 wt % water.

Said medical implant may include a non-fibrous filler. Such a filler may be included to reduce the risk of cracking of the medical implant. A said non-fibrous filler is preferably bio-compatible. It is preferably inert. It may be a colloidal particulate material. It may be swellable in water.

Said non-fibrous filler preferably comprises particles having an average particle size of less than 1 μm, more preferably less than 0.1 μm. Said non-fibrous filler preferably comprises colloidal particles.

Said non-fibrous filler preferably comprises a silica.

The ratio of the weight of hydrogel on a dry matter basis to the weight of non-fibrous filler may be at least 3, preferably at least 5, more preferably at least 7. Said ratio may be less than 20, suitably less than 15, preferably less than 14, more preferably less than 10.

The ratio of the weight of fibrous filler to non-fibrous filler, each on a dry matter basis, may be less than 10, suitably less than 8, preferably less than 5. The ratio may be at least 1.5, preferably at least 3.

The Young's Modulus of said medical implant measured in a linear elastic region, for example between 0.75 MPa and 1.0 MPa may be at least 15 MPa, is preferably at least 19 MPa and, more preferably, is at least 23 MPa. The Young's Modulus may be less than 50 MPa, or less than 30 MPa.

By selecting an implant having an elastic modulus as described, the implant may be compressed to fit within an opening during implantation and may expand to form an interference fit in the opening after release of the compressive force.

Said medical implant preferably has a coefficient of friction which is significantly less than that of natural cartilage. The medical implant may have a coefficient of friction of less than 0.1N, preferably less than 0.05N.

Said medical implant is preferably for use in repairing cartilage. It preferably comprises a cartilage plug which is suitably arranged to be received in an opening defined in cartilage for example cartilage which has been damaged by disease or wear.

Said cartilage plug preferably has a curved, preferably endless, outer wall which preferably extends around an axis. The outer wall is preferably substantially smooth, preferably across substantially its entire extent.

The outer wall is preferably substantially symmetrically arranged about said axis. The outer wall is preferably of substantially constant cross-section along its extent.

The length of said plug in the direction of said axis is suitably at least 5 mm, preferably at least 7 mm. The length may be less than 20 mm, preferably less than 15 mm, more preferably less than 12 mm, especially 10 mm or less. The maximum width of the plug in a direction perpendicular to said axis is suitably at least 2 mm, preferably at least 4 mm, more preferably at least 6 mm. The maximum width may be less than 25 mm, preferably less than 20 mm, more preferably less than 16 mm. The ratio of the maximum width to the length may be at least 0.5, preferably at least 0.7. Said ratio may be less than 2, preferably less than 1.6.

Said cartilage plug is preferably cylindrical. It may have a volume of at least 100 mm², preferably at least 200 mm². The volume may be less than 2000 mm². Said cartilage plug is preferably solid across substantially its entire extent. It preferably includes substantially no void areas.

Said medical implant may include chondrocytes which may be distributed, preferably substantially homogenously, throughout said hydrogel.

Said medical implant may include hyaluronic acid or a precursor or derivative thereof. Said acid, precursor or derivative is preferably arranged to improve the environment within the implant for chondrocyte growth. Said implant may include less than 1.5 wt %, preferably less than 1 wt %, more preferably less than 0.8 wt % of said acid, precursor or derivative. Said implant may include at least 0.1 wt %, preferably at least 0.3 wt %, more preferably at least 0.5 wt % of said acid, precursor or derivative.

Said medical implant may incorporate other active ingredients. For example, it may incorporate one or more antibiotics (e.g. gentomycin) and/or one or more anti-inflammatories (e.g. ibuprofen).

The medical implant may have a density of less than 2 g/ml, preferably less than 1.5 g/ml, more preferably less than 1 g/ml. The density may be greater than 0.5 g/ml.

According to a second aspect of the invention, there is provided a medical implant which incorporates hyaluronic acid or a precursor or derivative thereof.

The medical implant of the second aspect may have any feature of the medical implant of the first aspect.

According to a third aspect of the invention, there is provided a method of making a medical implant as described according to the first or second aspects, the method comprising selecting a mixture comprising a fibrous filler together with a hydrogel or precursor of a hydrogel, introducing said mixture into a mould shaped to define said implant, allowing the mixture to cure, and removing the cured mixture from said mould.

According to a fourth aspect, there is provided a method of treating or repairing a defect in a human body, the method comprising introducing an implant according to said first or second aspects into said human body in order to treat or repair the defect.

According to a fifth aspect, there is provided the use of an implant according to the first or second aspects for treating or repairing a defect in a human body.

According to a sixth aspect, there is provided a medical implant according to the first or second aspects for use in treating or repairing a defect in a human body.

The inventions of the fourth, fifth and sixth aspects preferably comprise treating or repairing a defect in cartilage in a human body. Such treatment or repair may comprise introducing the implant into an opening, for example a cylindrical opening, in the body. The opening may be defined in a surgical procedure which may comprise defining an opening within damaged cartilage of said body. Prior to introduction of the implant, it may be compressed, suitably to reduce its volume in at least one direction so that it can be introduced into the opening. Thereafter, the implant may expand, suitably to fill the opening, and suitably so that it is an interference fit therewithin. The implant may be associated with a joint. It may be an osteochondral implant. It may be used to treat or repair cartilage in a knee joint.

Any feature of any aspect of any invention or embodiment described herein may be combined with any feature of any aspect of any other invention or embodiment described herein mutatis mutandis.

Specific embodiments of the invention will now be described, by way of example, with reference to the accompanying drawings, in which:

FIG. 1 is a schematic representation of a knee joint as referred to above;

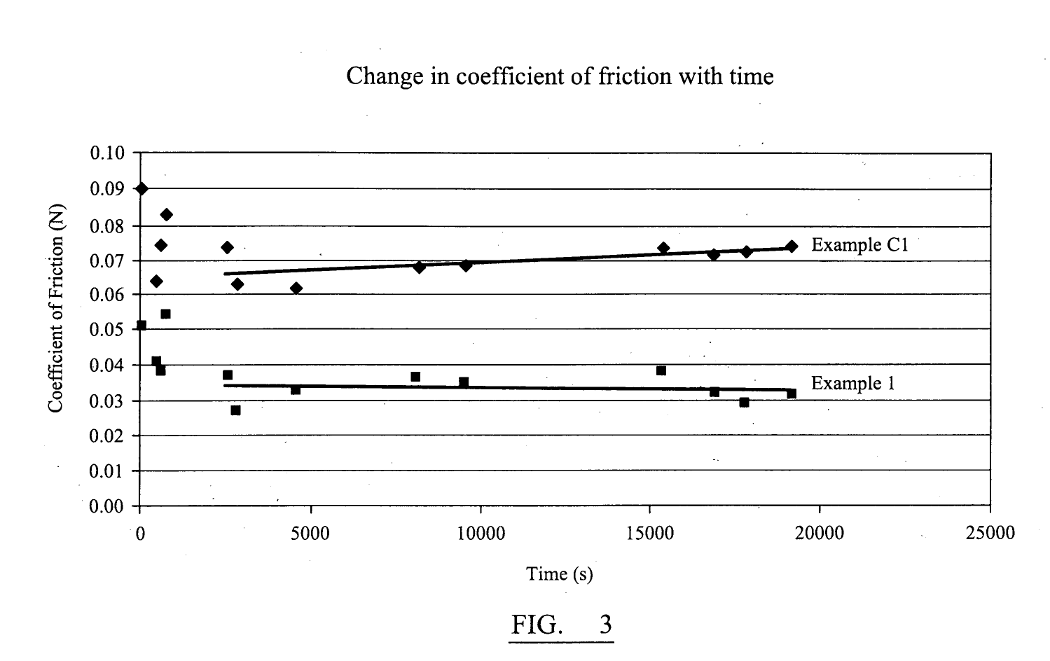

FIG. 2 shows a test piece which is used in assessing mechanical properties of different artificial cartilage plugs; and

FIG. 3 is a graph illustrating the change in coefficient of friction with time for plugs of example 1 and C1 respectively.

The following materials are referred to hereinafter:

Poval 220—a polyvinylalcohol obtained from Kuraray having a viscosity, measured on a 4% aqueous solution at 20° C. (determined by a Brookfield synchronised-meter rotary-type viscometer), of 30 .mPa·s and a degree of hydrolysis (saponification) of about 88% mol %. The molecular weight is about 130,000.

Cab-o-sil fumed silica M5. Untreated fumed silica obtained from Cabot Corporation.

PVA fibre—refers to fully hydrolysed PVA fibre.

Various different cartilage plugs were prepared for use in the procedure described above with reference to FIG. 1. The plus were subjected to relevant tests and compared to a commercially available plug.

### EXAMPLE 1

### Preparation of poly (1,4-di(4-(N-methylpyridinyl))-2,3-di(4-(1-formylphenyl)butylidene

This was prepared as described in Example 1 of PCT/GB97/02529, the contents of which are incorporated herein by reference. In the method, an aqueous solution of greater than 1 wt % of 4-(4-formylphenylethenyl)-1-methylpyridinium methosulphonate (SbQ) is prepared by mixing the SbQ with water at ambient temperature. Under such conditions, the SbQ molecules form aggregates. The solution was then exposed to ultraviolet light. This results in a photochemical reaction between the carbon-carbon double bonds of adjacent 4-(4-formylphenylethenyl)-1-methylpyridinium methosulphate molecules (I) in the aggregate, producing a polymer, poly (1,4-di(4-(N-methylpyridinyl))-2,3-di(4-(1-formylphenyl)butylidene methosulphonate (II), as shown in the reaction scheme below. It should be appreciated that the anions of compounds I and II have been omitted in the interests of clarity.

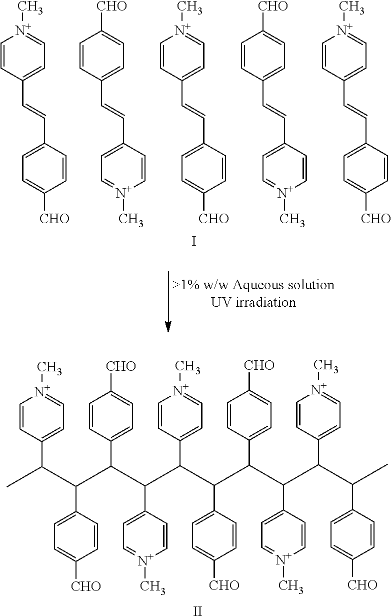

### EXAMPLE 2

### Preparation of Cartilage Plug

To 15 wt % aqueous solution of Poval 220 (15 g) at ambient temperature was added, with stirring, an 80 wt % concentrated solution of the butylidene polymer of Example to delivery 0.2 g of polymer on a dry matter basis together with dilute phosphoric acid (0.3 ml) to give a pH of 3.0. The ingredients were thoroughly mixed. To the mixture was added Cabosil (0.7 g) and the mixture thoroughly mixed at ambient temperature. After mixing PVA fibre (1.5 g) was added and the mixture mixed by hand until the ingredient had been thoroughly mixed. During mixing the mixture was relatively “putty-like”.

After mixing, the mixture was cast into a mould to define a cartilage plug and allowed to cure overnight under ambient conditions. The plug was then removed from the mould.

### EXAMPLE 3

### Modified Pin-on-Plate Wear Testing

Pin-on-plate wear testing is a widely used test to simulate the in-vivo wear characteristics and kinematics of articulating joints. This therefore presents the opportunity to assess the wear properties of two materials that come into contact under similar sliding speeds and contact stresses to those that occur in the human body. However, earlier tests showed that the standard test methodology creates unrealistic conditions and causes wear in radically different mechanisms than would be expected in articulating joints, and as a result tearing of the samples occurred. A modified, constrained pin-on-plate wear, test was developed which prevents tearing whilst still allowing the wear of the material to be monitored in a realistic environment.

To prepare samples for testing, fresh bovine cartilage was obtained from the femoral condyles of a bovine knee within 36 hours of slaughter. Cylindrical plugs 20 (FIG. 2) of 10 mm in diameter were drilled from the femoral condyles to a depth of 12 mm so as to include between 1.5-2.0 mm of cartilage 22 as well as subchondral and cancellous bone 24. Immediately prior to testing, 6.0 mm diameter holes were drilled to a depth of 8.5 mm to accommodate 6.5 mm diameter plugs 26. Note that the plug is compressed slightly during insertion into the hole.

A linear, multi-station test rig (Plint & Partners Ltd, UK) was set up to operate at 1 Hz across a sliding distance of ±25 mm about a central position for up to 191k cycles. The samples were secured in specimen holders, which in turn were secured at one end of a loaded arm. The arm was loaded to produce a contact stress of 1 MPa between the faces of the plugs and a flat stainless steel plate with a surface roughness of Rₐ=0.02 μm. The plugs were aligned perpendicular to the surface of the stainless steel plate to ensure uniform stress and corresponding wear. The loaded arm contained calibrated strain gauges to record sliding forces, thereby allowing a coefficient of friction to be calculated and continuously monitored. A bovine serum-distilled water mix of 30-70% respectively was used as a lubricant. Testing occurred at room temperature (18° C.), although the operating temperature of the lubricant bath reached a stable 27° C. during testing.

In addition, the Young's Modulus of the plugs was determined by calculation from stress-strain unloading curve in linear elastic regions of samples

### Results and Discussion

The cartilage plug of example 2 was tested as described in Example 3 and compared to a commercially available plug (referred to as Example C1)

After an initial 25.2k cycles of wear (7 hours) testing was suspended and the Example 1 and C1 samples analysed. In both cases the level of cartilage 22 was below the level of the plug 26—this was particularly evident in the case of Example C1 which was deemed to have failed. Evidence of directional wear was apparent on the cartilage under a light microscope but not on the plugs of the Example 1 or C1 samples. In the case of Example C1 curling of the plug 26 around the external circumference of the cartilage 22 occurred and this was much more apparent than for the Example 1 sample. Continued degradation of the cartilage occurred with further wear. This was particularly evident with the Example C1 sample after a total of 81k cycles. After 81k cycles the Example C1 plug protrudes from the surface of the sample by approximately 1.5 mm compared to 0.5 mm for the Example 1 plug. Although cartilage was still present around the Example C1 plug the subchondral bone was visible through it. Substantial curling of cartilage is apparent in comparison to a relatively small amount of curling with the Example 1 sample.

Subsequent testing up to 107k cycles shows acceleration in the wear of the cartilage in both samples but more significantly with the Example C1 sample. This resulted in an increase in protrusion of the Example C1 plug from the face of the cartilage. At this stage a gap around the circumference of the Example C1 plug had become visible. The cartilage layer was particularly thin around one of the leading edges of the Example C1 plug and immediately around the circumference of the plug itself. In the case of the Example 1 plug cartilage degradation continued but had uniform thickness across the whole of the cartilage.

After termination of the test at 191k cycles no wear debris was visible on the surface of either of the Example 1 or C1 samples. Both plugs protruded above the face of the cartilage/subchondral bone. The cartilage had almost entirely worn away in the case of the Example C1 sample and 0.5 mm thickness remained with the Example 1 sample. A gap of approximately 0.4 mm was visible around the Example C1 plug. Both the protrusion and the gap around the Example C1 plug allowed it to easily slide out of the subchondral bone. In contrast, the Example 1 plug had to be gouged out using tweezers resulting in destructive damage to the plug.

An overall reduction in length of 0.21 mm was recorded for the Example C1 plug and no detectable change in length could be measured for the Example 1 plug.

Both materials showed an initial drop in friction over the first few thousand cycles. Fitting a least mean square linear regression to the data from 3000 cycles onwards (see FIG. 3) shows a trend towards a decreasing friction with an increase in number of cycles for the Example 1 plug but an increase in friction for the Example C1 plug over the same number of cycles.

Youngs Modulus values were obtained from the unloading curve between 0.75 MPa and 1.0 MPa (a linear elastic region) and values of 26.5 MPa and 26.3 MPa were obtained for the Example 1 plug. The Example C1 plug had a value of 5 MPa. The Young's Modulus of cartilage and bone is 15.6 MPa.

When implanted in a human body and juxtaposed adjacent living cartilage it may be expected that chondrocytes may migrate into an artificial cartilage plug. In fact, this may be desirable since it may enable Type II collagen to be made within the plugs. However, it is of course important that properties of the plug are not significantly detrimentally affected by the presence of chondrocytes. Example 4 describes features of plugs impregnated with chondrocytes.

### EXAMPLE 4

### Hydrogel Plugs Impregnated with Chondrocytes

Plugs were prepared as described in Example 1 except that at the initial mixing stage 1 ml of a chondrocyte formulation (containing 5×10⁶ chondrocytes per ml) prepared by culturing in a bioreactor, were introduced. Plugs were prepared with live chondrocytes. It was found that the plugs had very similar performance to that described in Example 1. The Young's Modulus was found in two replicated tests to be 36.6 mPa and 39.0 mPa.

It is desirable to encourage integration of artificial cartilage plugs with existing cartilage when implanted in a human body. In order to improve the environment within the artificial plugs for chondrocyte growth and production by it of Type II collagen, hyaluronic acid may be incorporated into plugs as described in the following example.

### EXAMPLE 5

### Hydrogel Plugs Impregnated with Hyaluronic Acid

Following the procedure described in Example 1 plugs were prepared using the following ingredients.

15 wt % aqueous solution of Poval 220 (10 g)

hyaluronic acid (0.08 g)

butylidene polymer of Example 1 (0.15 g)

Cabosil (0.2 g)

Phosphoric acid (0.1 ml) (Concentrated acid diluted with an equal volume of water)

PVA fibre (0.8 g)

Plugs prepared were assessed and found to have mechanical properties which surpass those of the Examples 1 and C1 embodiment. The plug of Example 5 was found to have a Young's Modulus of 26.5 mPa.

It should now be appreciated that plugs according to preferred embodiments have properties which surpass those of commercially available materials. Tests suggest their performance in situ will be significantly improved compared to such commercially available materials.

1. A medical implant comprising a polymeric material and a fibrous filler.

2. An implant according to claim 1, said implant including at least 65 wt % water and said polymeric material comprising a hydrogel.

3. An implant according to claim 2, wherein said hydrogel comprises an optionally derivatised hydrophilic polymer.

4. An implant according to claim 3, where said hydrophilic polymer is selected from polymethacrylic acid polymers, polyimides, polyvinylalcohol and copolymers of any of the aforesaid.

5. An implant according to claim 3, wherein said hydrophilic polymer includes both carbonyl moieties and hydroxyl moieties.

6. An implant according to claim 2, wherein said hydrogel comprises an optionally derivatised polyvinylalcohol.

7. An implant according to claim 2, wherein said hydrophilic polymer of said hydrogel is cross-linked by a cross-linking means which preferably comprises a second polymeric material which includes a repeat unit of formula 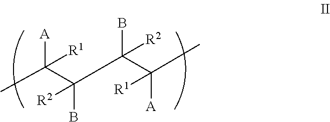
wherein A and B are the same or different, are selected from optionally-substituted aromatic and heteroaromatic groups and at least one comprises a relatively polar atom or group and R¹ and R² independently comprise relatively non-polar atoms or groups.

8. An implant according to claim 1, wherein said fibrous filler comprises an optionally cross-linked hydrophilic polymer.

9. An implant according to claim 8, wherein said hydrogel includes an optionally cross-linked hydrophilic polymer and said fibrous filler comprises the same optionally cross-linked hydrophilic polymer.

10. An implant according to claim 1, wherein said fibrous filler comprises optionally derivatised polyvinylalcohol fibres.

11. An implant according to claim 8, wherein the ratio of the weight of hydrogel on a dry-matter basis to the weight of fibrous filler on a dry-matter basis is at least 1.

12. An implant according to claim 2, wherein the wt % of hydrogel in said medical implant on a dry-matter basis is at least 40 wt % and less than 80 wt % and the weight % of fibrous filler in said medical implant on a dry-matter basis is at least 10 wt % and is less than 40 wt.

13. An implant according to claim 1, which includes a non-hydrous filler, for example silica.

14. An implant according to claim 1, wherein the Youngs Modulus of said medical implant in a linear elastic region, for example between 0.75 MPa and 1.0 MPa, is at least 15 MPa; and the medical implant has a coefficient of friction of less than 0.1 N.

15. An implant according to claim 1 which comprises a cartilage plug.

16. An implant according to claim 1 which includes chondrocytes.

17. An implant according to claim 1, which includes hyaluronic acid or a precursor or derivative thereof.

18. A medical implant which incorporates hyaluronic acid or a precursor or derivative thereof.

19. A method of making a medical implant according to claim 1, for example for replacing defective cartilage, the method comprising selecting a mixture comprising a fibrous filler together with a hydrogel or precursor of a hydrogel, introducing said mixture into a mould shaped to define said implant, allowing the mixture to cure, and removing the cured mixture from said mould.

20. A method of treating or repairing a defect in a human body, for example for replacing defective cartilage, the method comprising introducing an implant according to claim 1 into said human body in order to treat or repair the defect.

21. The use of an implant according to claim 1 for treating or repairing a defect in a human body.

22. A medical implant according to claim 1 for use in treating or repairing a defect in a human body, for example for replacing defective cartilage.
